(19) United States
(12) Patent Application Publication
(10) Pub. No.: US 2004/0006493 A1
(43) Pub. Date: Jan. 8, 2004

(54) Eyewear education method and system

(76) Inventor: Edward L. Flaherty III, Wexford, PA (US)

Correspondence Address: Pepper Hamilton LLP, One Mellon Center, 50th Floor, 500 Grant Street, Pittsburgh, PA, 15219, US

(21) Appl. No.: 10/365,871

(22) Filed: Feb. 13, 2003

Related U.S. Application Data
(60) Provisional application No. 60/356,316, filed on Feb. 13, 2002.

(51) Int. Cl. G06F 17/60; A61B 3/00

(52) U.S. Cl. 705/2; 351/200

(57) ABSTRACT

An eyewear education method and system educates a patient or customer seeking consultation with an eye care professional about various eyewear options. An eyewear education form is provided to the patient/customer prior to or during the patient's/customer's consultation with the eye care professional. The customer is instructed to read the form, which is comprised of various options available for choosing eyewear, and then instructed to fill in the blanks on the form, checking off his/her personal preferences for the various eyewear options presented. Optionally, the customer may also be requested to fill out personal information about himself/herself. The form is then presented to the eye care professional prior to or during the customer's consultation with the eye care professional.

### PRIORITY

This application claims priority to the provisional U.S. patent application entitled, Eyewear Education Method and System, filed Feb. 13, 2002, having a Ser. No. 60/356,316, the disclosure and drawings of which are hereby incorporated by reference.

### FIELD OF THE INVENTION

The present invention relates generally to an improved method and system for educating a customer or patient seeking consultation with an eye care professional about various eyewear options. More particularly, the present invention relates to a method and system of informing the patient or customer, prior to or during an examination with an eye care professional, about various options of eyewear available.

### BACKGROUND OF THE INVENTION

Currently, when patients or customers who seek the advice of an eye care professional enter the professional's office, the patient or customer has no easy way of knowing the various options for eyewear that may be available. Instead, the patient/customer must either perform independent research before coming in to the office, in which case the patient/customer still cannot be sure that he or she has completely discovered all available options, or the patient/customer must wait for the professional to explain the options, which not only is an inefficient use of consultation time, but also often leaves little or no time for consideration and decision making relating to the options. Whether the professional is an ophthalmologist, optometrist, optician, or other eye care professional, the need for an improved method and system of educating an eye care patient or customer about various eyewear options just prior to meeting with the eye care professional is highly desirable.

Accordingly, it is desirable to provide an improved method and system for educating an eye care patient or customer about various eyewear options.

### SUMMARY OF THE INVENTION

It is therefore a feature and advantage of the present invention to provide an improved method and system for educating an eye care patient or customer about various eyewear options. In accordance with a preferred embodiment of the present invention, an Eyewear Education Update form is provided to the patient or customer prior to or during the patient's/customer's consultation with the eye care professional. The customer is then instructed to read the form, which is comprised of various options available for choosing eyewear. By way of example, such options may include, but are not limited to, providing information relating to the following: frame style such as traditional, semi-rimless, and rimless; frame material such as plastic, metal, stainless steel, titanium, and flexible metal frames; lens prescription such as single vision, bifocal lenses, trifocal lenses, and progressive lenses; lens material such as glass, plastic, high-index plastic, polycarbonate, and trivex lenses; and lens color such as clear, clear lenses that change, fixed tint, sunglasses, clip-on sunglasses, and click-on sunglasses. Optionally, the options may include information regarding polarization, anti-reflective coating, scratch resistant coating, and UV 4 filter. The customer may be instructed to fill in blanks in the form or to check off his/her personal preferences for the various eyewear options presented. Optionally, the customer may also be requested to fill out personal information about himself/herself, such as where the customer spends time, a description of the customer's personal interests, and a qualification of the customer's activity level. The form is then presented to the eye care professional prior to or during the customer's consultation with the eye care professional.

There have thus been outlined the more important features of the invention in order that the detailed description that follows may be better understood, and in order that the present contribution to the art may be better appreciated. There are, of course, additional features of the invention that will be described below and which will form the subject matter of the claims appended hereto.

In this respect, before explaining at least one embodiment of the invention in detail, it is to be understood that the invention is not limited in its application to the details of construction and to the arrangements of the components set forth in the description or illustrated in the drawings. The invention is capable of other embodiments and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be used as a basis for designing other structures, methods, and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is a block diagram that illustrates the steps that may be followed in accordance with a preferred embodiment of the present invention.

FIG. 2 illustrates a first page of an exemplary patient/customer information form.

FIG. 3 illustrates a second page of an exemplary patient/customer information form.

### DETAILED DESCRIPTION OF PREFERRED EMBODIMENTS OF THE INVENTION

A preferred embodiment of the present invention provides an improved method and system for educating an eye care patient or customer about various eyewear options. In accordance with a preferred embodiment of invention, an Eyewear Education Update (“E.E.U.”) form is provided to a patient or customer prior to the consultation with the eye care professional. The form may be of any format, but in a preferred embodiment the form is one double-sided page.

The E.E.U. is intended to inform the patient or customer of the various options available when choosing his/her eyewear. The office manager or receptionist can give the E.E.U. to the patient/customer when the patient/customer arrives for his/her appointment, or it can also be filled out by the eye care professional as the patient/customer is in consultation with the eye care professional. Optionally, the E.E.U. may be sent out as a direct mail piece to the patient/customer from the eye care professional prior to the patient's/customer's visit.

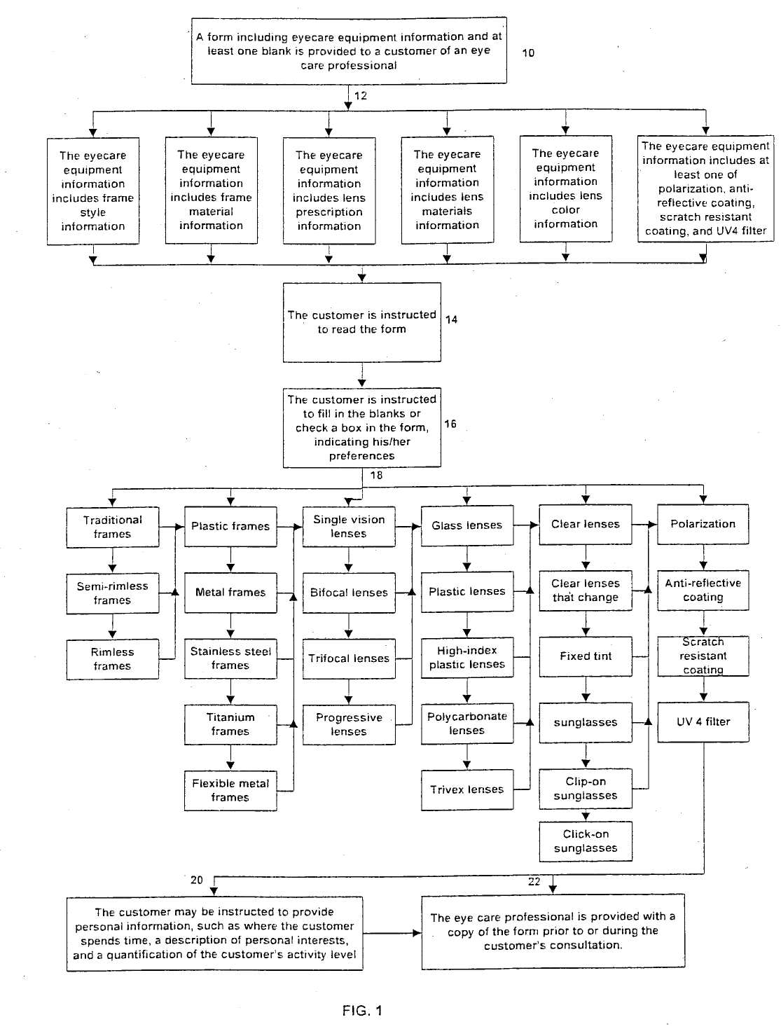

In a preferred embodiment, an eye care patient or customer is educated about various eyewear options in accordance with steps such as those illustrated in FIG. I. Referring to FIG. 1, a form including eyecare equipment information and at least one blank is provided to a customer of an eye care professional (step 10). Optionally, the eyecare equipment information may include any one or more of the following: frame style information, frame material information, lens prescription information, lens materials information, lens color information, and information about polarization, anti-reflective coating, scratch resistant coating, and/or UV 4 filters (step 12). The customer is then instructed to read the form (step 14), and to either fill in the blanks or check a box in the form indicating his/her preferences (step 16). A preferred embodiment allows the customer to choose from the following frame style information: traditional frames, semi-rimless frames, rimless frames; the following frame material information: plastic frames, metal frames, stainless steel frames, titanium frames, flexible metal frames; the following lens prescription information: single vision lenses, bifocal lenses, trifocal lenses, progressive lenses; the following lens materials information: glass lenses, plastic lenses, high-index plastic lenses, polycarbonate lenses, trivex lenses; the following lens color information: clear lenses, clear lenses that change, fixed tint, sunglasses, clip-on sunglasses, click-on sunglasses; and one or more of the following: polarization, anti-reflective coating, scratch resistant coating, and UV 4 filter (step 18). Optionally, the customer may be instructed to provide personal information, such as where the costumer spends time, a description of personal interests, and a quantification of the customer's activity level (step 20). Finally, the eye care professional is provided with a copy of the form prior to or during the customer's consultation.

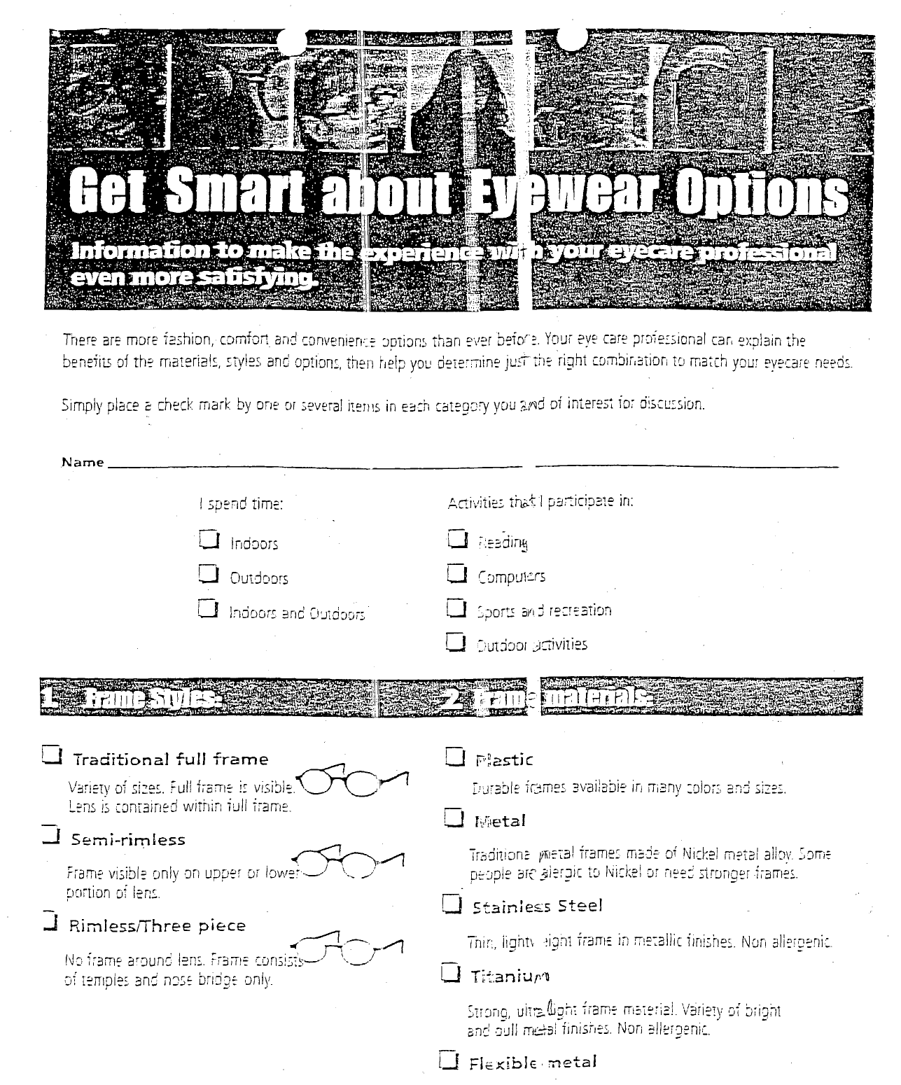

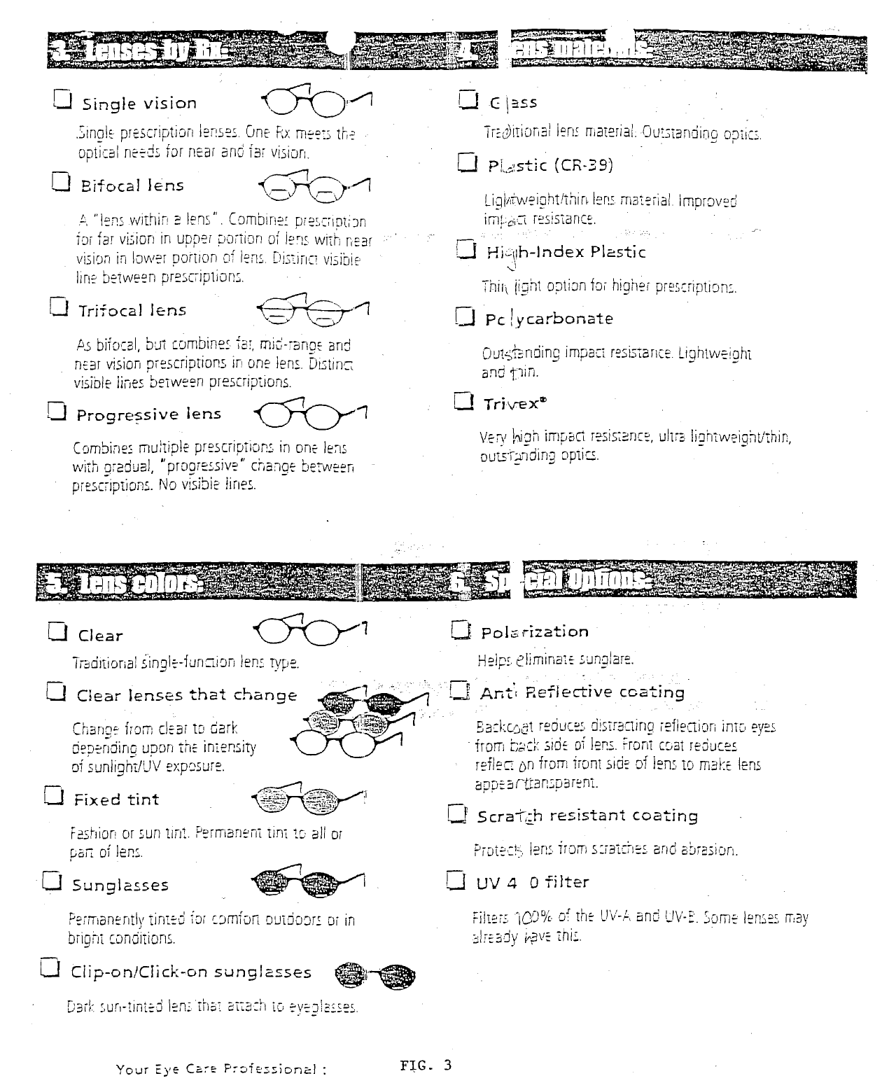

A preferred embodiment of the patient/customer information form is illustrated in FIGS. 2 and 3. As illustrated in FIGS. 2 and 3, the form may advise the patient/customer about options relating to some or all of the following areas, among others: frame styles, frame materials, lens types by prescription, lens types by materials, lens colors, and other options such as glare reduction, filters, and coatings. Optionally, the form may ask for personal information about the patient/customer, such as the patient's/customer's activity levels and interests, and whether the patient/customer is more likely to spend time indoors or outdoors.

When the patient/customer has completed the form, the form is presented to the eye care professional. The eye care professional will then be informed about the patient/customer's individual preferences regarding the various eyewear options. Additionally, by using the form, the patient/customer will have participated more intimately in the process of selecting his/her eyewear. Thus, the informed patient/customer will have a more meaningful discussion with the eye care provider in determining the best eyewear solution. Ultimately, an informed discussion with the consumer who has read the E.E.U. has the potential to yield higher levels of patient/customer satisfaction and potentially high sales per transaction for the eye care professional.

The many features and advantages of the invention are apparent from the detailed specification. Thus, the appended claims are intended to cover all such features and advantages of the invention which fall within the true spirits and scope of the invention. Further, since numerous modifications and variations will readily occur to those skilled in the art, it is not desired to limit the invention to the exact construction and operation illustrated and described. Accordingly, all appropriate modifications and equivalents may be included within the scope of the invention.

What is claimed is:

1. A method of educating a customer of an eye care professional about eyewear options, comprising the steps of:
providing a form to the customer at any time prior to or during the customer's consultation with the eye care professional, wherein the form includes eye care equipment information and at least one blank for the customer to fill in;
instructing the customer to read the form;
instructing the customer to fill in the blanks in the form; and
providing the eye care professional with a copy of the form prior to the customer's consultation.

2. The method of claim 1, wherein the eye care equipment information includes frame style information.

3. The method of claim 2, wherein the frame style information comprises a description of at least one of traditional, semi-rimless, and rimless frames.

4. The method of claim 1, wherein the eye care equipment information includes frame material information.

5. The method of claim 4, wherein the frame material information comprises a description of at least one of plastic, metal, stainless steel, titanium, and flexible metal frames.

6. The method of claim 1, wherein the eye care equipment information includes lens prescription information.

7. The method of claim 6, wherein the lens prescription information comprises a description of at least one of single vision, bifocal lens, trifocal lens, and progressive lenses.

8. The method of claim 1, wherein the eye care equipment information includes lens materials information.

9. The method of claim 8, wherein the lens materials information comprises a description of at least one of glass, plastic, high-index plastic, polycarbonate, and trivex lenses.

10. The method of claim 1, wherein the eye care equipment information includes lens color information.

11. The method of claim 10, wherein the lens color information comprises a description of at least one of clear, clear lenses that change, fixed tint, sunglasses, clip-on sunglasses, and click-on sunglasses.

12. The method of claim 1, wherein the eye care equipment information includes at least one of polarization, anti-reflective coating, scratch resistant coating, and UV 4 filter.

13. The method of claim 1, wherein the blank consists of a check box corresponding to the eye care information.

14. The method of claim 1, further comprising the step of instructing the customer to provide personal information.

15. The method of claim 14, wherein the personal information comprises information regarding at least one of where the customer spends time, a description of the customer's personal interests, and a quantification of the customer's activity level.

16. A system for educating a customer to consultation with an eye care professional about eyewear options, comprising a form, wherein the form comprises:
eye care information comprising information regarding at least one of frame style, frame material, and lenses;
at least one blank for the customer to fill in, wherein the blank consists of a check box corresponding to the eye care information; and
a request for personal information from the customer, wherein the personal information comprises information regarding at least one of where the customer spends time, a description of the customer's personal interests, and a quantification of the customer's activity level.

17. The system of claim 16, wherein the information regarding frame style comprises a description of at least one of traditional, semi-rimless, and rimless frames.

18. The system of claim 16, wherein the information regarding frame material comprises a description of at least one of plastic, metal, stainless steel, titanium, and flexible metal frames.

19. The system of claim 16, wherein the information regarding lenses comprises:
lens prescription information comprising a description of at least one of single vision, bifocal lens, trifocal lens, and progressive lenses;
lens materials information comprising a description of at least one of glass, plastic, high index plastic, polycarbonate, and trivex lenses; and
lens color information comprising a description of at least one of clear, clear lenses that change, fixed tint, sunglasses, clip-on sunglasses, and click-on sunglasses.

20. The system of claim 16, wherein the eye care information further comprises a description of at least one of polarization, anti-reflective coating, scratch resistant coating, and UV 4 filter.
